(19) United States
(12) Patent Application Publication
(10) Pub. No.: US 2018/0201948 A1
(43) Pub. Date: Jul. 19, 2018

(54) DROUGHT TOLERANT PLANT SYSTEM

(71) Applicants: Sandra Isabel González Morales, Irapuato (MX); Luis R. Herrera-Estrella, Irapuato (MX)

(72) Inventors: Sandra Isabel González Morales, Irapuato (MX); Luis R. Herrera-Estrella, Irapuato (MX)

(21) Appl. No.: 15/838,342

(22) Filed: Dec. 11, 2017

Related U.S. Application Data
(60) Provisional application No. 62/432,502, filed on Dec. 9, 2016.

(51) Int. Cl. C12N 15/82; C07K 14/415

(52) U.S. Cl. CPC C12N 15/8273; C12N 15/8237; C07K 14/415

(57) ABSTRACT

A drought resistant plant system, including transgenic plants, methods of conferring drought resistance on a plant, and methods of cultivating a plant.

### CROSS-REFERENCE TO RELATED APPLICATION

This application is based upon and claims the benefit under 35 U.S.C. § 119(e) of U.S. Provisional Patent Application Ser. No. 62/432,502, filed Dec. 9, 2016, which is incorporated herein by reference in its entirety for all purposes.

### INTRODUCTION

Desiccation tolerance (DT) is the ability to survive the extreme loss of most (e.g., 95%) cellular water without accumulation of lethal damage. DT is considered to be a complex trait key in the conquering of dry land. DT in vegetative tissues is relatively common in less complex plants such as bryophytes and lichens, rare in pteridophytes and angiosperms, and absent in gymnosperms. In angiosperms, DT is rare in whole plants (vegetative tissues) but present in reproductive structures (pollen and seeds). It has been speculated that DT in vegetative tissue was the ancestral state for early land plants (e.g., mosses), which was lost early in the evolution of tracheophytes.

The seed is a key structure in the plant life cycle that helps the dispersal and survival of the species. Traits such as DT are important in this respect. DT is acquired during the seed maturation phase, which involves a complex regulatory network. In Arabidopsis, LEC1, LEC2, FUS3, and ABI3 are transcription factors (TFs) that are key players in seed maturation including DT; their mutants lack DT which have absent or reduced some components like LEA and heat shock proteins and the accumulation of oligosaccharides.

Although these TFs are essential in control of seed maturation, their overexpression in the background of these mutants is not able to restore tolerance to desiccation in seeds.

### SUMMARY

This disclosure presents a drought resistant plant system, including transgenic plants, methods of conferring drought resistance on a plant, and methods of cultivating a plant.

### BRIEF DESCRIPTION OF THE DRAWINGS

FIGS. 1a-c show a global gene expression profile in desiccation intolerant seed mutants. FIG. 1 a is a heatmap from hierarchical clustering of differentially expressed genes. Expression patterns of differential genes identified in at least one of the evaluated lines; each comparison was performed against the respective wild type (lec1-1 and lec2-1 versus ws; abi3-5 versus ler; fus3-3 versus col0) at three development stages (15, 17, and 21 DAF). Green indicates down-regulated values, red indicates up-regulated values, and black indicates unchanged values. The yellow squares represent the common downregulated genes between intolerant mutants FIG. 1b is a set of Venn diagrams showing the number and distribution of differential genes across the comparison among mutants represent down-regulated genes at 15, 17, and 21 DAF, respectively. Genes related with desiccation tolerance are indicated in red. FIG. 1c shows enriched GO Terms in Genes Upregulated and Downregulated during seed desiccation tolerance. The network graphs show BiNGO visualization of overrepresented GO terms for desiccation tolerance-specific genes either downregulated (1331 genes) during desiccation seed. Categories in GO Terms were used, including Cellular Components (CC), Biological Processes (BP), and Molecular Functions (MF). Uncolored nodes are not overrepresented, but they may be the parents of overrepresented terms. Colored nodes represent GO terms that are significantly overrepresented (Benjamini and Hochberg corrected P value<0.05), the color node indicates significance as shown in the color bar. A more detailed analysis of the GO categories is shown in Supplemental Table 25. ER, endoplasmic reticulum.

FIG. 2 shows a proposed metabolic pathway of carbohydrate metabolism in Arabidopsis seeds. Graphs represents changes in the contents of carbohydrates during desiccation period. Values lines indicate the log2fold change relative content compared to wild type seeds. Values are representative of two independently grown set plants and are presented as the mean of three biological repetition of 50 mg of isolated seed bulked from a least 10 plant for each stage. For statistic analysis and absolute content seed Supplemental Table 26. Heatmaps represent expression profiles of putative enzymes involved in raffinose and stachyose synthesis as well as sucrose degradation pathways. Red and green represent upregulated and downregulated genes, respectively.

ALKALINE/NEUTRAL INVERTASE (A/N-INVB, AT4G34860; A/N-INVD, AT1G22650), CYTOSOLIC INVERTASE (CINV2, AT4G09510 ; CINV1, AT1G35580), CELL WALL INVERTASE 1 (CWINV1, AT3G13790), SUCROSE SYNTHASE (SUS1, AT5G20830; SUS2, AT5G49190; SUS3, AT4G02280), UDP-D-GLUCOSE/UDP-D-GALACTOSE 4-EPIMERASE (UGE1, AT1G12780; UGE2, AT4G23920; UGE3, AT1G63180 ; UGE4, AT1G64440; UGES, AT4G10960), GALACTINOL SYNTHASE (GOLS1, AT2G47180; GOLS2, AT1G56600; GOLS3, AT1G09350; GOLS4, AT1G60470 ; GOLS5, AT5G23790; GOLS10, AT5G30500), RAFFINOSE SYNTHASE (RS1, AT1G55740; RS2, AT3G57520; RS5, AT5G40390; RS6, AT5G20250), STACHYOSE SYNTHASE (STS, AT4G01970).

FIGS. 3a-c show the TFsSeedNet and Subnetworks. FIG. 3a is an overview of the TFsSeedNet obtained at DP10.0. The lower insets show subnetworks of TFs present in downregulated genes. FIG. 3b shows snetDT1, which is related to nutrient storage. FIG. 3c shows snetDT2, which is related to cellular protection mechanisms. Both subnetworks, snetDT1 and snetDT2, were obtained from FullSeedNet at DPI 0.1. Genes are represented as nodes and inferred interactions as edge. The square shows nodes attributes. Nodes are colored green, represent downregulated genes. Edge width and color intensity is proportional to the Mutual Information (MI) value of the interaction, with higher MI values corresponding to thicker and darker edges. The shape of the node represents the development stage, triangle at 15DAF, and circle at 17 and 21 DAF. The border node represent the categories.

FIGS. 4a-f show that transcriptomic network inference reveals novel genes involved in desiccation tolerance. FIG. 4a shows identification of mutants affected in desiccation tolerance phenotype. Germination percentages of seeds 6 days after imbibition. Germination percentages were counted at different time points after imbibition. Values are the means and standard deviations (SD) of four technical replicates with 100 seeds per replicate. FIGS. 4b-d show PLATZ1 overexpression rescues abi3-5 intolerance desiccation phenotype. In particular, FIG. 4c shows phenotype of three representative lines from abi3-5/35S::PLATZ1 showing rescues of germination FIG. 4d shows mature seed from ler, abi3-5, and abi3-5/35::PLATZ1 TFs overexpressers were stored 0, 2, and 4 weeks followed by stratification and germination on 0.5X MS plates to assess desiccation tolerance. Values are means j SEM of three biological replicates. FIG. 4e shows how neighboring genes of PLATZ1 from snetDT2 are related to cellular protection mechanisms obtained from FullSeedNet at DPI 0.1. FIG. 4f shows qPCRs from 35::PLATZ1 from neighboring gene.

### DETAILED DESCRIPTION

The present disclosure provides a drought tolerant plant system. It is based on the study of the regulatory networks controlling desiccation tolerance in plant seeds. Using Arabidopsis mutants, this disclosure identifies two networks that control most if not all of the genes involved in desiccation tolerance. As part of these networks, the disclosure identifies several transcription factor genes that act as major nodes of the regulatory networks. Several of these are previously uncharacterized, and others have been studied before but not as part of these regulatory networks. Expressing some or all of these transcription factor genes in vegetative tissues generates a significant degree of drought tolerance. The present disclosure seeks protection for the previously uncharacterized genes (isolated and in transgenic plants), the use of the individual genes or combinations of TF genes that are part of the networks that regulate desiccation tolerance to confer water stress tolerance, and the use of the genes to confer tolerance to other stresses such as high salt or cold. The disclosure further seeks to protect methods of using the plant system, and components thereof, in research and agriculture.

Through comparative analysis of RNA-seq and metabolic profiles of lec1, lec2, fus3, and abi3 mutants, as well as their corresponding wild types during seed desiccation period, we identified expressed genes specifically involved in the DT process. The associated data enabled us to integrate metabolic processes, signaling pathways, and specific TF activity. Additionally, we showed that reverse engineering of a DT-specific regulatory network reveals transcriptional modules that activate the DT genes in seeds. Notably, two major transcriptional networks were identified related to storage of reserve compounds and cellular protection mechanisms, respectively. Ectopic expression of some TFs identified in these subnetworks is sufficient to activate genes that contribute to DT because it partially rescues the desiccation intolerance phenotype of abi3 mutant, whereas the elimination of these TFs showed a reduced seed desiccation tolerance.

The TFs identified in both subnetworks are potentially regulators of DT. If these TFs are key to confer the DT, the TFs will confer drought tolerance to vegetative tissues. Hence, the present disclosure provides a transgenic plant engineered to have increased drought tolerance or increased water use efficiency. The disclosure may include use of individual TFs, or combinations of TFs, that control the subnetworks that regulate the establishment of desiccation tolerance in seeds.

Further aspects of the present disclosure are described in the following sections: (I) system overview, and (II) selected embodiments. The “Supplementary Figures” and “Supplementary Tables” mentioned in these sections are contained in U.S. Provisional Patent Application Ser. No. 62/432,502, filed Dec. 9, 2016, which is incorporated herein by reference in its entirety for all purposes.

### I. System Overview

Desiccation tolerance (DT) is an amazing process that allows seeds in the dry state to remain viable for periods that can reach hundreds or even thousands of years'⁻³. It has been postulated that seed DT evolved by rewiring the regulatory and signaling networks that controlled vegetative DT that had emerged as a crucial adaptive trait of the early land plants. Phylogenetic analyses suggest that vegetative DT was initially present in less complex plants, such as bryophytes, but was then lost in the evolution of vascular plants, when this complex adaptive trait was rewired to be active during seed rather than vegetative tissues^{4,5}. Interestingly, at least eight independent cases of evolution (or re-evolution) of vegetative DT have occurred in the angiosperms and one in gymnosperms⁴. The independent re-evolution of vegetative DT in different angiosperm clades suggests that, despite being quite complex processes, both vegetative and seed DT might be controlled by one or few regulatory networks composed of a discrete number of transcription factors (TFs). Understanding the networks that regulate seed DT in model plant systems will provide the tools to understand an evolutionary process that played a crucial role in the diversification of the plant kingdom. This disclosure presents an integrated approach involving genomics, bioinformatics, metabolomics, and molecular genetics to identify and validate molecular networks that control the acquisition of DT in Arabidopsis seeds.

Desiccation tolerance (DT) may be defined as an ability to survive the loss of most (e.g., 95%) cellular water without accumulation of lethal damage. DT organisms orchestrate a complex number of responses to protect cellular structures and prevent damage to proteins and nucleic acids. Early land plants evolved mechanisms to survive harsh drying environments that allowed them successfully to exploit different ecosystems in the land. Therefore, it has been postulated that the initial evolution of vegetative DT, in both vegetative and reproductive stages, was a crucial step required for the colonization of land by primitive plants from a fresh water origin⁶.

Seed DT, a trait that allows terrestrial plants to survive long periods of lack of water until favorable conditions are present for germination, is probably part of the answer to Darwin's “abominable mystery”: the sudden appearance of great angiosperm diversity in the fossil record. In angiosperms, DT is acquired at the seed maturation stage, which involves a complex regulatory network^{7,8} that activates a large subset of genes involved in a number of mechanisms that influence seed survival in the dry state. The set of genes required for seed DT includes genes encoding protective proteins, such as late embryogenesis abundant (LEA)^{9,16} and heat shock proteins (HSPs)¹¹, enzymes involved in scavenging reactive oxygen species¹², and the biosynthesis of protective compounds, such as oligosaccharides^{7,13}, and antioxidants, such as tocopherols and flavonoids^{14,16}.

In Arabidopsis, embryo development and seed maturation, including the acquisition of DT, is orchestrated by a set of four master regulators: LEAFY COTYLEDON 1 (LEC1), which is a CCAAT-box binding factor, and three B3 domain-containing proteins¹⁶, ABSCISIC ACID INSENSITIVE 3 (ABI3), FUSCA 3 (FUS3), and LEC2. In addition to controlling embryo formation and seed maturation, these master regulators also repress the expression of genes required for the transition from embryo to vegetative development¹⁷⁻²⁰. Although the role of these master regulators on seed maturation is globally similar, some of their functions are specific; for example, in contrast to mutations in LEC1, ABI3, and FUS3 that drastically affect DT^{21,22}, mutations in lec2 do not affect DT^{22,23}. Interestingly, ectopic expression of LEC1, FUS3, or ABI3 in single or double mutant backgrounds of the other two regulators activate some processes of seed maturation, such as lipid and seed storage protein accumulation, but not DT, suggesting that all three regulators are required to activate DT. Genetic evidence suggests that downstream of LEC1, FUS3, ABI3, and LEC2 other TFs play important roles in integrating the subnetwork that regulates specific aspects of embryo development and seed maturation and in particular seed DT²⁴. Although several processes involved in seed maturation and their regulatory mechanisms have been studied in Arabidopsis²⁵ and Medicago^{7,8} the regulatory networks activating the DT process remain largely unknown.

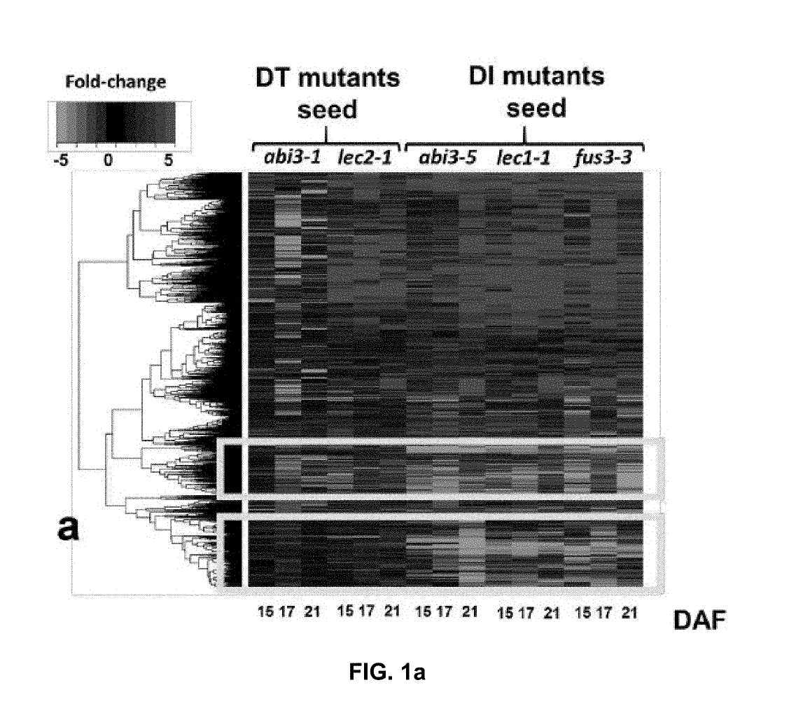

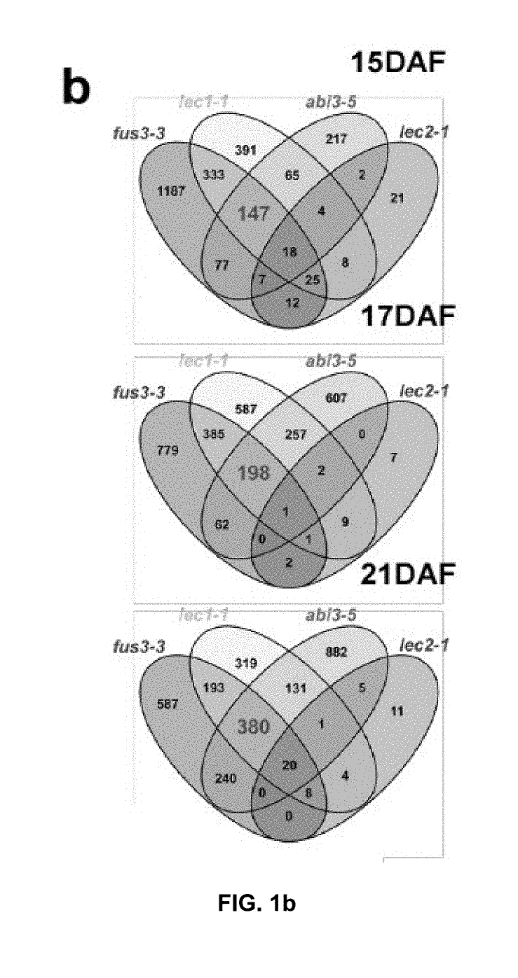

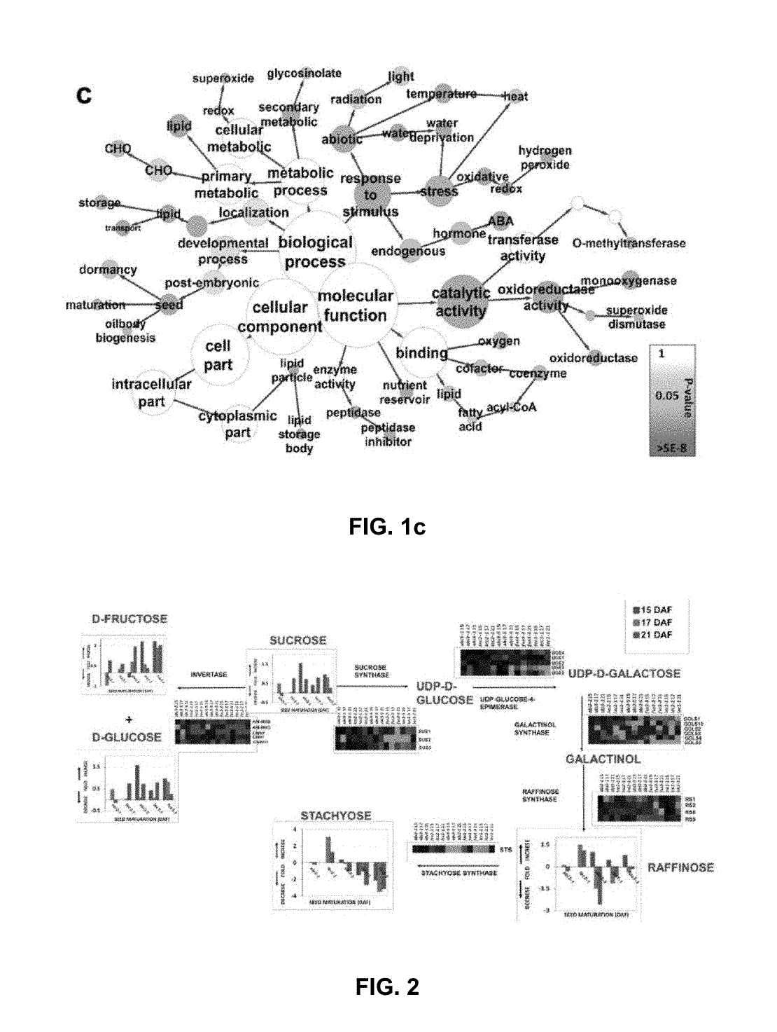

To identify the regulatory subnetworks involved in the acquisition of DT that act downstream of LEC1, FUS3, and ABI3, we designed a comparative transcriptomic analysis between the seed desiccation intolerant (DI) lines lec1-1, abi3-5, and fus3-3 and DT lines lec 2-1 and the corresponding wild type controls for each mutant line. In this comparative analysis, we also included abi3-1, a DT weak allele of abi3²³. Since lec1 and lec2 have similar phenotypes, including morphological alterations during embryo development and reduced accumulation of storage compounds, but differ in DT, a comparative analysis should allow the identification of genes that are directly involved in DT and that are activated in lec2 but not in lec1 (Supplementary Table 1). To have a global view of the transcriptional differences between DI and DT lines during seed maturation, we constructed libraries for RNA-sequencing for each of the analyzed lines at three developmental stages (Supplementary Table 2), specifically, 15 DAF (days after flowering), a developmental stage previous to drastic water loss, 17 DAF, when rapid water loss starts, and 21 DAF, when the seed is completely dried (Supplementary FIG. 1). To determine differentially expressed genes (DEGs), RNASeq data was analyzed using two types of model analysis: (1) a generalized linear model (GLM) based on an interaction model (Supplementary Tables 3-8); and (2) a GLM based on pairwise comparison26 (Supplementary Tables 1 and 9-23). Applying a stringency level of false discovery rate (FDR) of <0.05 and log 4-fold change (log2FC)≥1, we identified 3450 differentially expressed genes (DEGs) in at least one of the three developmental stages sampled for this analysis. Among the DEGs, 2218 were upregulated (Supplementary Tables 3-5) and 1331 were downregulated by the tolerance effect (Supplementary Tables 6-8). We also identified differences between tolerant and intolerant Arabidopsis mutants at each of the tested developmental stages (FIGS. 1a-c; Supplementary Tables 9-23).

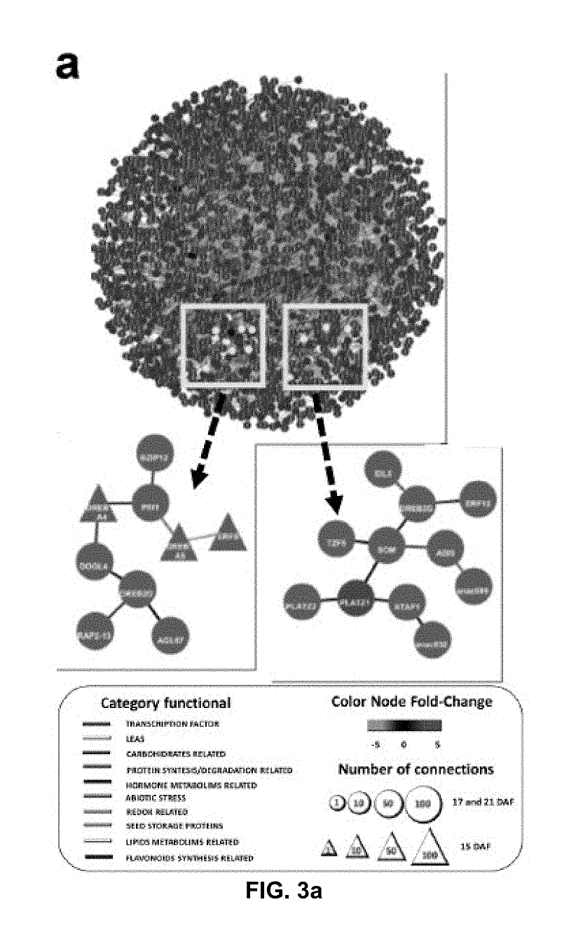

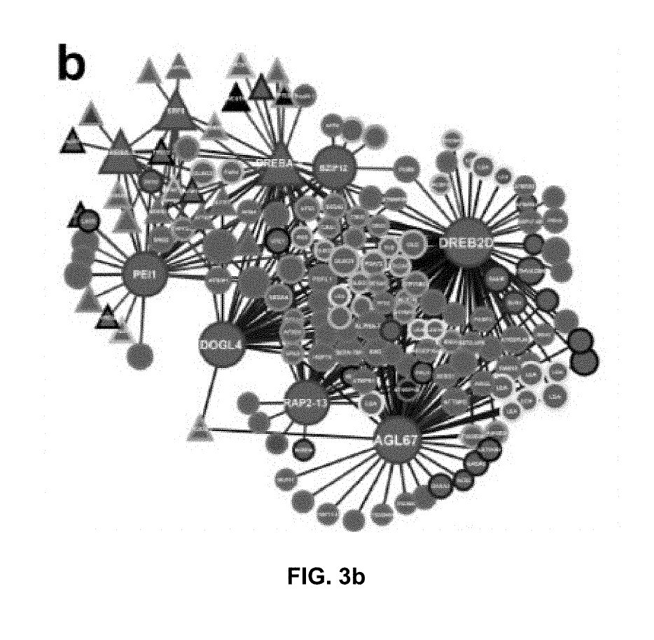

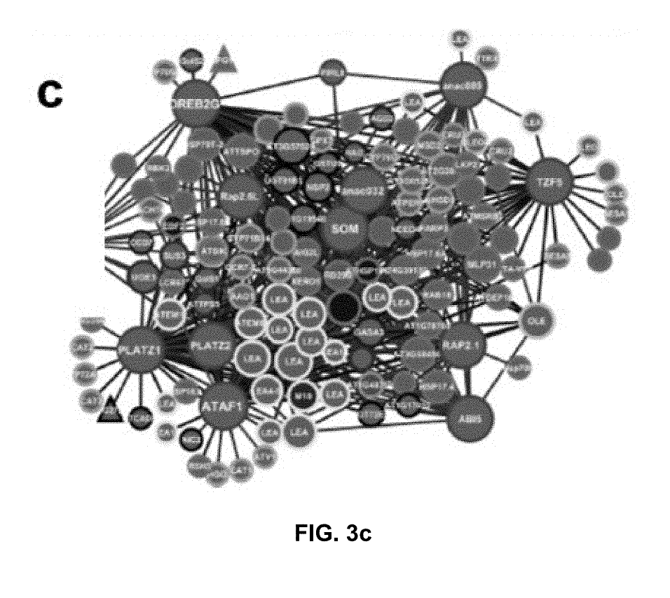

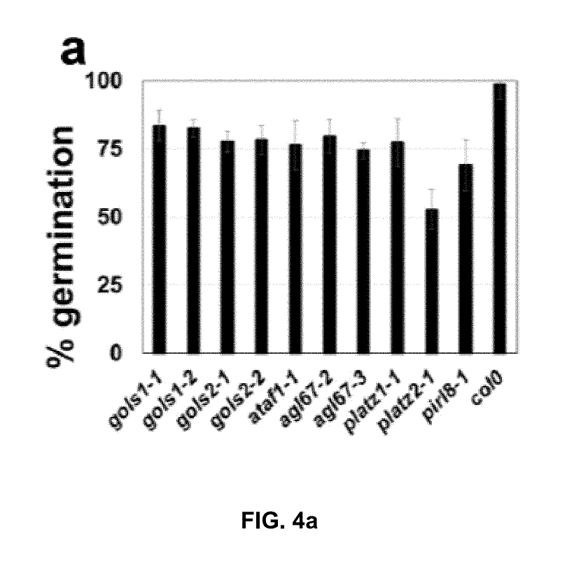

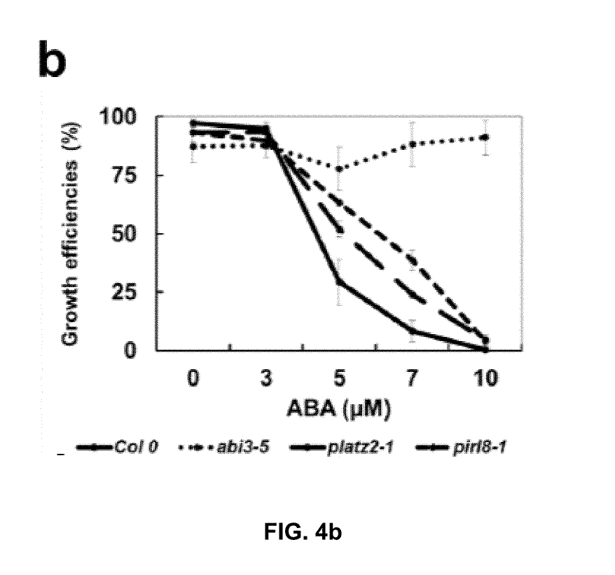

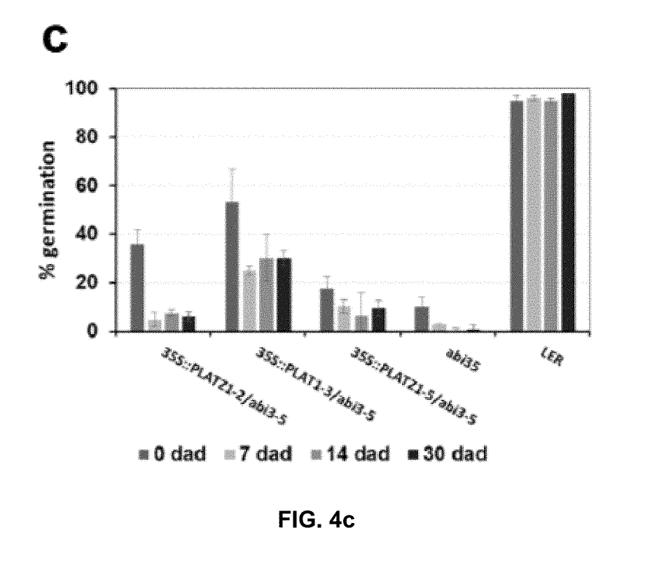

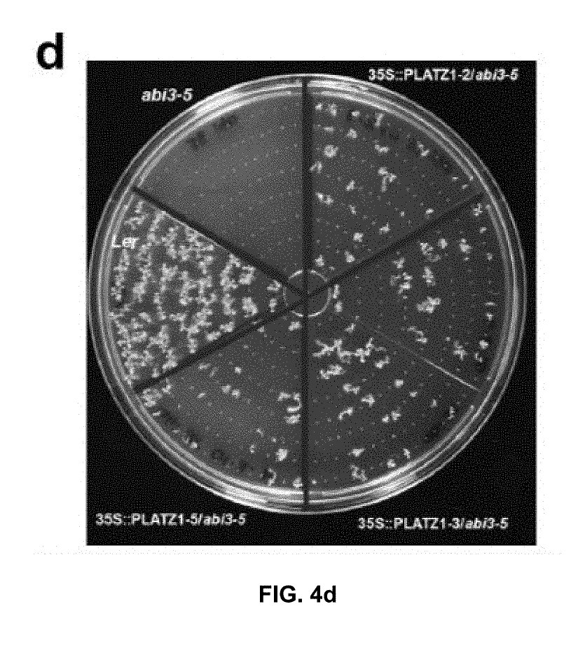

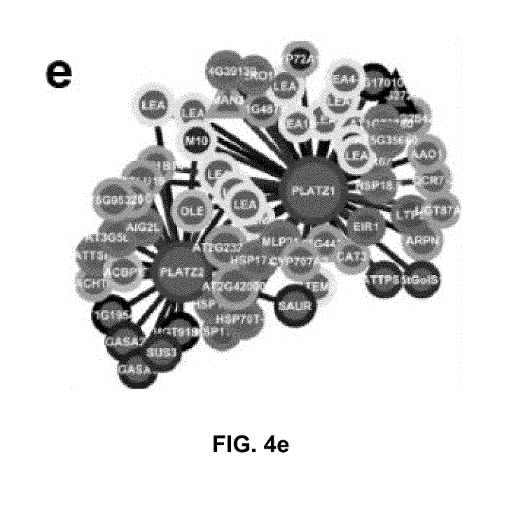

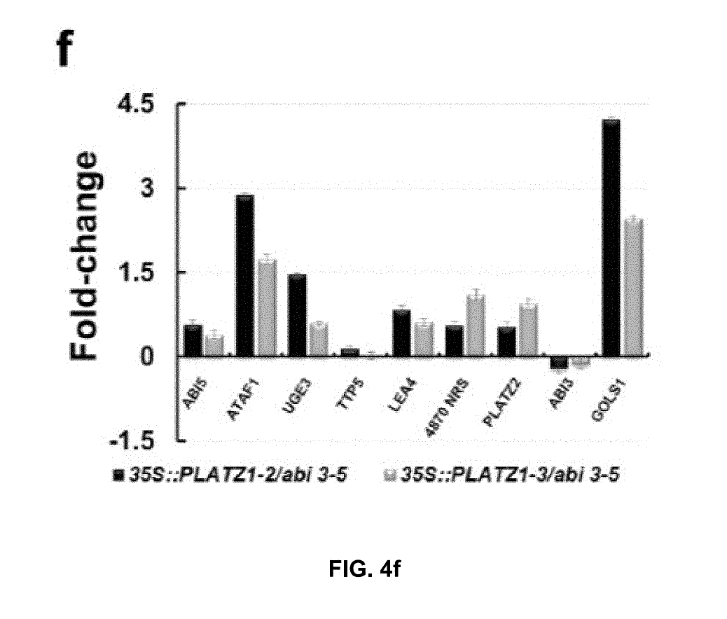

The heatmap of DEGs shows that a large subset of genes are upregulated in lec2-1, lec1-1, abi3-5, and fus3-3, relative to their wild-type (WT) controls, probably representing genes that are activated as part of the direct transition of these mutants from embryo to vegetative growth rather that entering into dormancy and DT (FIG. 1a). This group of genes is not activated in abi3-1 and includes genes involved in chloroplast activity, such as photosystem (PS) I and II, carbon fixation, chlorophyll (tetrapyrrole) biosynthesis, and amino acid metabolisms (Supplementary FIG. 2; Supplementary Text).

The heatmap also shows a second subset of DEGs, which transcription fails to activate in all DI mutants with respect to their DT controls and which appear as drastically repressed in the heatmap, probably representing genes that are directly or indirectly relevant for the acquisition of DT in Arabidopsis seed. The set of downregulated DEGs specific for DI mutants increased as the level of water content decreased in the seed (FIG. 1b). Downregulated DEGs specific for DI mutants were enriched in the following gene ontology (GO) categories: (1) in molecular function: oxidoreductase activity and nutrient reservoir; (2) in biological process: stress responses, lipid and carbohydrate biosynthesis, and seed development. In responses to stimulus, abscisic acid (ABA), and stress responses, such as water, oxidative, and temperature, were significantly enriched (FIG. 1c; Supplementary Table 25). A MAPMAN classification of the same set of genes showed enrichment in categories such as abiotic stress, LEA protein synthesis, and metabolic pathways including rafinosa, stachyose, and trehalose biosynthesis (Supplementary FIG. 3). The finding that overrepresented categories of downregulated genes specific for DI lines were enriched in GO and MAPMAN categories related to stress responses and cell protection categories and that these categories were more clearly defined at 17 and 21 DAF, when the seeds were in the rapid process of water loss, confirmed that DI mutants fail to activate mechanisms required to acquire DT in the seed. Interestingly, lec2-1 has a much lower number of downregulated genes than lec1-1, fus3-1, and abi3-5, which correlates well with the fact that these mutants are still capable of acquiring DT (Supplementary FIG. 5b,c).

To complement our transcriptional study, carbohydrate profiles of DT and DI mutants were determined using the same seed developmental stages chosen for the RNASeq analyses (15, 17, and 21 DAF). We specifically analyzed the raffinose-related soluble carbohydrate sucrose, stachyose, and raffinose. This analysis showed that in lec1-1, abi3-5, fus3-3, and lec2-1, sucrose levels at 15 DAF were one-fold higher than their WT controls, which later decreased to almost WT level at 21 DAF, whereas raffinose levels decreased 1.5- and 2.5-fold in abi3-5 and 1- and 0.7-fold in lec1-1 at 17 and 21 DAF, respectively, while in lec2-1 raffinose increased two-fold at 21 DAF.

Stachyose decreased three-fold in lec1-1 and fus3-3, whereas lec2-1 had a three-fold increase with respect to the wild type control (FIG. 2; Supplementary Table 26). In agreement with a reduction in content, the transcript levels of genes encoding key enzymes in these oligosaccharides, such as sucrose synthases (AT5G4919, SUS2, and AT4G02280, SUS3), UDP-D-galactose-4-epimerases (AT1G63180, UGE3 and AT4G10960, UGE5), galactinol synthases (AT2G4718, GOLS1 and AT1G09350, GOLS2), and stachyose synthase (AT4G01970, STS) were strongly repressed (FIG. 2). In Arabidopsis, the accumulation of stachyose and raffinose has been proposed as a key factor in the acquisition of DT, acting as “water replacement” providing the hydrogen bonds required for membrane and protein stabilization, as well as protecting DNA against hydrolytic damage¹³.

To predict transcriptional regulatory networks responsible for establishing seed DT in Arabidopsis, we constructed co-expression regulatory networks using two curated datasets obtained from 169 seed-specific CEL files from 23 ATH1 microarray experiments (Supplementary Table 27). We produced a general co-expression regulatory network of all genes expressed during seed development (FullSeedNet) (Supplementary FIG. 7, Supplementary Table 29) and a TF only co-expression network of TF genes expressed during seed development (TFsSeedNet)²⁷ (FIG. 3a; Supplementary Table 28). To identify TFs potentially involved in regulating the establishment of seed DT in Arabidopsis, we integrated the TF genes differentially expressed in DI mutants in the TFsSeedNet (Supplementary Tables 6-8). We found that these TFs clustered into two different groups: (1) upregulated genes (red) on one side of the global TF seed network, and (2) downregulated genes (green) on the opposite side (FIG. 3a). These results confirm our prediction that, depending on the increased or decreased expression in DI mutants, differentially expressed TFs participate in distinct developmental processes. Specifically, upregulated genes are involved in processes related to the embryo to vegetative transition, whereas downregulated genes are involved in metabolic processes related to stress tolerance and/or the storage of reserve molecules.

The repressed TF genes specific for DI mutants (genes that are activated and potentially participate in the acquisition of DT but that fail to be activated in DI mutants) formed two main co-expression subnetworks, which we termed snetDT1 and snetDT2 (FIGS. 4d,e, Supplementary FIGS. 8 and 9). snetDT1 was composed mainly of members of the AP2-ERP TF gene family (AT1G75490, AT1G22190, AT4G31060, AT1G01250), but also contained members of the bZIP (AT2G41070), MADS-BOX (AGL67), C2H2 (PE11; AT5G07500), and DOG-like (DOGL4; AT4G18650) TF families. As part of the TFs that integrate snetDT2, we identified two members of the NAC family (ATAF1; AT1G01720 and ANAC089; AT5G22290), two of the AP2-ERBP family (DREB2G; AT5G18450 and ERF12; AT1G28360), two of the C3H family (SOM; AT1G03790 and TZF5; AT1G03790), two of the PLATZ family (PLATZ1; AT1G21000; and PLATZ2; AT1G76590), and one of the BZIP family (ABI5; AT2G36270). Here, PLATZ refers to “plant AT-rich sequence- and zinc-binding protein.”

We then searched in FullSeedNet for the non-TF genes that are co-expressed with the snetDT1 and snetDT2 TFs and that represent their putative target for transcriptional activation. snetDT1 was integrated by a total of 280 genes (FIG. 4d; Supplementary Table 30), from which the most significant enriched categories included development storage protein, lipid metabolism, and abiotic stress (Supplementary FIG. 10, Supplementary Table 32). snetDT1 starts to be detectable 15 DAF and is integrated by three major TF nodes (DOGL4, PEI1, and DREBA-4) that interact with genes mainly involved in lipid metabolism and the accumulation of storage proteins. Among the genes forming part of snetDT1 with strong interactions (MI>0.5 and p-value<1e-35; Supplementary Table 27) at 15 DAF, we found fatty acid triacylglycerol (TAG) biosynthesis genes and storage protein genes, such as those encoding albumin proteins SESA1, SESA2, SESA3, SESA4, and SESAS, cruciferin proteins CRU2 and CRU3, and oleosin proteins OLE1, OLE2, and OLE4. Seventeen DAF, DOGL-4 is maintained as a major node in snetDT1; however, three other TFs (DREB2D, AGL67, and RAP2-13) are incorporated as major nodes in this subnetwork. DREB2D (AT1G5490) appears to be a key TF in snetDT1 because it has the largest number of interactions and it is strongly co-expressed (MI>0.6 and p-value<1E-40) with other TFs such as AGL67 and DOGL4. These three TFs have as potential targets genes related to stress tolerance, including 11 LEA genes (Supplementary FIG. 8; Supplementary Tables 30 and 32).

The second subnetwork (snetDT2) was composed of 317 genes, which represent 17% of all downregulated genes from time-specific tolerance differences (FIG. 4e; Supplementary FIG. 9). snetDT2 is enriched in genes in the following categories: redox activity, LEA protein genes, and carbohydrate (CHO) metabolism (including raffinose metabolism) (Supplementary Tables 31 and 33). In snetDT2, we identified ATAF1 as a TF with an elevated number of interactions (81), which is strongly co-expressed (MI>0.5 and p-values<1E-35) with other TFs, such as PLATZ1, SOM, and DREB2G. These four TFs are central in snetDT2 because each has a large number of interactions and shares common nodes that correspond to genes involved in cellular protection. For example, among the genes involved in stress resistance that are potentially activated by these TFs, we found 3 LEA group 4 genes (LEA4-1, LEA4-5, and LEA4-2), two LEA group 1 genes (ATEM1 and ATEM6), three genes involved in raffinose synthesis (UGE3, SUS3, GOLS1), two catalase genes (CAT2, CAT3), one superoxide dismutase (SOD), two small heat shock proteins (HSP17.4 and HSP17.6), and two genes involved in ABA signaling (PP2C and ABI5) (FIG. 3; Supplementary FIG. 9; Supplementary Tables 31 and 32). Interestingly, snetDT2 was specifically activated at 17 DAF and became more complex at 21 DAF, which corresponds to the developmental stages at which rapid, or total, water loss occurs (FIG. 4e; Supplementary FIG. 9). ATAF1, a NAC TF, was the node with highest number of interactions in snetDT2, which potentially targets stress protection genes. We identified three other TFs with a high number of interactions, namely, SOM, DREB2G, and PLATZ1/2, which also preferentially interacted with genes associated with stress protection processes (FIG. 4e).

We then searched for enriched cis-regulatory elements in the promoters of target genes in each subnetwork. In general, a large number of enriched motifs were detected with ABA signaling-related (ABF binding site motif, ABRE binding site motif, ACGT ABRE motif A2OSE). The seed specific motif (RY-repeat promoter motif) was found in the three stages of snetDT1, whereas dehydration and drought responses (ABRE-like binding site motif, DRE core motif, CBF1 BS in cor15a, AtMYC2 BS in RD22) were enriched in snetDT2 (Supplementary Table 34). This finding supports our model in which at early stages snetDT1 regulates seed filling genes and at later stages snetDT2 regulates DT genes (Supplementary FIG. 17).

If the genes identified as major nodes in snetDT2 indeed play a role in DT, it would be expected that mutation in some of these genes would lead to some degree of DI. We therefore tested T-DNA insertion mutants in PLATZ1, PLATZ2, AGL67, DREB2D, DREB4-A, and ATAF1 to determine whether insertions reduced germination percentage as a consequence of a decreased DT. It was observed that the germination rate of ATAF1, AGL67, PLATZ1, and PLATZ2 was reduced by 76, 75, 77, and 53%, respectively (FIG. 4a). Interestingly, mutant seed of PIRL8 that belongs to a plant-specific class of intracellular LRRs that likely mediate protein interactions, possibly in the context of signal transduction²⁸, also had a decreased germination and ABA insensibility (FIG. 4b) This gene is connected with TFs SOM, DREB2G, and ANAC089, which have high connections with genes involved in cellular protection mechanism (FIG. 3e).

The TFs identified as major nodes in the DT subnetworks must regulate the expression of target genes directly involved in DT, such as those involved in oligosaccharide biosynthesis or encoding LEA proteins. To determine whether some snetDT2 non-TF genes have a role in DT, we determined the germination phenotype of their T-DNA insertion mutants. We found that GOLS1 and GOLS2 (enzymes involved in raffinose synthesis) had a reduction in germination of 20 to 30% with respect to the WT control (FIG. 4a). These results suggest that some of the target genes of the major nodes of snetDT2 indeed play important roles in DT.

If some of the TFs identified as major nodes in snetDT1 and snetDT2 act downstream of ABI3, FUS3, and LEC1 and play an important role in activating effector genes involved in DT, overexpression of these TFs in DI mutants, such as abi3-5, should partially revert the desiccation intolerance phenotype of these mutants. To test this, we expressed AGL67, DREB2A, and DREB4A from snetDT1 and PLATZ1, PLATZ2, and DREB2G from snetDT2, under control of the 35S promoter of the cauliflower mosaic virus in the abi3-5 background (FIGS. 4c,d). Dry seeds were collected and stored for 1, 2, and 4 weeks and then tested for germination efficiency. As previously reported, abi3-5 seed rapidly losses viability after desiccation and after 2 weeks of storage germination was reduced to less than 10%. In contrast, seed from abi3-5/35S::PLATZ1, abi3-5/35S::AGL67, and abi3-5/35S::DREB2G lines showed a germination rate of 25% , 30%, and 12%, respectively, after 4 weeks of storage (FIG. 4c,d; Supplementary FIG. 11). These results show that ectopic expression of these TF genes partially rescued DT in abi3-5 seed. To determine whether the putative target genes are indeed transcriptionally activated by these TFs, we evaluated the effect of the 35S::PLATZ1 gene construct in the abi3-5 genetic background on the expression of some of the putative target genes of PLATZ1 (FIGS. 4e,f). Using two independent PLATZ1 overexpressing lines, we found that in most cases the putative target genes had a 1.5- to 4-fold higher expression in the PLATZ1 overexpressing lines than in the abi3-5 control line. These results show that the expression of the genes predicted to be activated by PLATZ1 is indeed directly or indirectly activated by this TF.

Our results suggest that PLATZ1 is capable of activating a subset of snetDT2 genes that seems to be important for the acquisition of DT in Arabidopsis seed. To test whether the activated genes indeed play an important role in DT, we introduced the 35S::PLATZ1 gene construct in the WT Arabidopsis Col 0 ecotype. Three-week-old plants of the 35S::PLATZ1 transgenic lines grown under full irrigation were subjected to a period of seven days without irrigation and the number of surviving plants scored 4 days after a recovery irrigation treatment. The four tested lines showed a 70 to 80% survival compared to a 10% survival recorded for the WT control (Supplementary FIG. 12).

In this disclosure, we provide direct evidence that demonstrates the importance of snetDT1 and snetDT2, and the TF that are major nodes in these subnetworks in the acquisition of DT in Arabidopsis seeds. The prediction of ARACNE about the potential targets of the TF identified as major regulatory nodes in snetDT1 and snetDT2, were confirmed by the observation that the expression of several of the putative targets of PLATZ1 are indeed upregulated in PLATZ1 overexpressing lines. A number of previously published reports support our conclusion: (1) Some of the TFs that are major nodes in these subnetworks seem to be directly activated by LEC1; for instance, it has been shown that PEI1 and DREB-A4 are activated by ectopic expression of LEC1²⁹; (2) overexpression in Arabidopsis of ATAF1 resulted in enhanced drought tolerance in Arabidopsis³⁰; (3) SOM is a TF belonging to the CCCH-type zinc finger protein that has been reported to negatively regulate seed germination by activating ABA biosynthesis and inhibiting GA biosynthesis³¹; and (4) although the precise function of DREB2G is still unknown, it belongs to a TF gene family that is generally involved in abiotic stress tolerance.

The DT is an ancestral feature and has evolved at least eight times in the angiosperms, suggesting a conserved regulation during the plant evolution. In our model, we proposed that as part of their fundamental role in embryo development and seed maturation program, LEC1, FUS3, and ABI3 activate the expression of a set of TFs that specifically act as regulators of DT in seeds via the direct or indirect activation of DT effector genes (Supplementary FIG. 17). LEC1 and ABI3 are highly conserved during evolution from bryophytes to angiosperms; thus, an important question is whether vegetative DT was originally control by this TF, which was then recruited to implement seed development or whether the networks responsible of DT were control by other master regulators specifically responsible of activation DT genes in an inducible manner in vegetative tissues.

Another interesting question is whether the regulatory subnetworks that control vegetative DT in basal plants are similar to those controlling DT in seeds. Toward this end, we performed a phylogenetic analysis of the conservation of the key nodes of snetDT1 and 2 networks. We found that PLATZ1, PLATZ2, AGL67, DREB2D, and DREB2G are conserved in the bryophytes Physcomitrella patents, in vascular DT basal plants such as Selaginella moellendorffii, in the basal angiosperm Amborella trichopoda, in the monocotyledonous species Oryza sativa (rice), Zea maize (corn) and DT plant Oropetium thomaeum, and in the dicotyledonous species Glycine max (soybean) and Solanum lycopersicum (tomato) (Supplementary FIGS. 13-16). This opens the possibility that a core DT regulatory network could have been conserved throughout plant evolution. Further research will be needed to explore whether DT is orchestrated by regulatory networks in which at least a common core of TFs has been conserved during plant evolution and determine how it has been rewired several time to be activated in seeds and in vegetative tissues. It is interesting that several of the nodes of snetDT 1 and 2 have seed-specific expression in different plants species; for example, PLATZ1 orthologues are specifically expressed during seed maturation in rice and soybean, and in maize in addition to being expressed during seed maturation show a high induction during drought stress (Supplementary FIG. 12). Finally, this disclosure presents the important biotechnological application of activating the expression of one or more of the members of these snetDT1 and 2 in vegetative tissue, by gene transfer or genome editing, to confer inducible drought tolerance in crop plants.

### II. Selected Embodiments

This section describes selected embodiments of the present disclosure, presented as a series of indexed paragraphs.

1. A transgenic plant, comprising a plant expressing in vegetative tissues an exogenous gene, wherein the exogenous gene encodes a transcription factor that confers drought resistance on the plant.

2. The transgenic plant of paragraph 1, wherein the exogenous gene is involved in seed development or production in wild-type plants.

3. The transgenic plant of paragraph 2, wherein the exogenous gene is involved in seed desiccation in wild-type plants.

4. The transgenic plant of paragraph 1, wherein the transcription factor is selected from the group consisting of PLATZ1, PLATZ2, ANAC089, ERF12, and AGL67.

5. The transgenic plant of paragraph 4, wherein the transcription factor is PLATZ1.

6. The transgenic plant of any of paragraphs 1-5, wherein the exogenous gene is introduced by gene transfer.

7. The transgenic plant of any of paragraphs 1-5, wherein the exogenous gene is introduced by genome editing.

8. The transgenic plant of paragraph 4, wherein the expression of the endogenous genes orthologue to one or more transcription factors from the group consisting of PLATZ1, PLATZ2, ANAC089, ERF12, and AGL67 is activated in vegetative tissues by genome editing.

9. The transgenic plant of paragraph 1, the exogenous gene being a first exogenous gene, the plant further expressing a second exogenous gene, the second exogenous gene also conferring drought resistance on the plant.

10. The transgenic plant of paragraph 9, wherein the first exogenous gene and the second exogenous gene encode distinct transcription factors.

11. The transgenic plant of paragraph 10, wherein each distinct transcription factor is selected from the group consisting of PLATZ1, PLATZ2, ANAC089, ERF12, and AGL67.

12. The transgenic plant of paragraph 11, wherein one of the distinct transcription factors is PLATZ1.

13. The transgenic plant of any of paragraphs 1-12, further comprising a constitutive promoter that controls expression of the exogenous gene.

14. The transgenic plant of paragraph 13, wherein the constitutive promoter is from a cauliflower mosaic virus.

15. The transgenic plant of any of paragraphs 1-12, further comprising an inducible promoter that controls expression of the exogenous gene, wherein the inducible promoter causes expression of the exogenous gene when the transgenic plant is experiencing water stress.

16. The transgenic plant of paragraph 1, further comprising a modification of the promoter that controls expression of the endogenous gene orthologous to PLATZ1, PLATZ2, ANAC089, ERF12, and AGL67, wherein the modification of the promoter causes expression of the endogenous gene when the transgenic plant is experiencing water stress.

17. A method of conferring drought resistance on a plant, comprising modifying cells of the plant to constitutively express a gene encoding a gene product that is normally expressed mainly during seed production.

18. The method of paragraph 17, wherein the gene product is a transcription factor.

19. The method of paragraph 18, wherein the transcription factor is selected from the group consisting of PLATZ1, PLATZ2, ANAC089, ERF12, and AGL67.

20. The method of paragraph 19, wherein the transcription factor is PLATZ1.

21. The method of any of paragraphs 18-20, wherein the transcription factor is from Arabidopsis thaliana.

22. The method of any of paragraphs 17-21, wherein the gene is an exogenous gene.

23. The method of any of paragraphs 17-22, wherein modifying cells of the plant includes introducing a constitutive promoter into the cells upstream from the gene, such that the gene is constitutively expressed.

24. The method of paragraph 23, wherein the constitutive promoter is an exogenous promoter.

25. The method of paragraph 24, wherein the constitutive promoter is from a cauliflower mosaic virus.

26. The method of any of paragraphs 17-22, wherein modifying cells of the plant includes introducing an inducible promoter into the cells upstream from the gene, such that the gene is inducibly expressed when the transgenic plant is experiencing water stress.

27. The transgenic plant of paragraph 17, wherein modifying cells of the plant includes a modification by genome editing of the promoter of the endogenous genes orthologue to one or more transcription factors from the group consisting of PLATZ1, PLATZ2, ANAC089, ERF12, and AGL67 that activates the expression of the transcription factor in vegetative tissues.

28. The method of paragraph 17, wherein modifying cells of the plant includes a modification of the promoter that controls expression of the endogenous gene orthologous to PLATZ1, PLATZ2, ANAC089, ERF12, and AGL67, wherein the modification of the promoter causes expression of the endogenous gene when the transgenic plant is experiencing water stress.

29. The method of paragraph 17, wherein the gene product leads to the accumulation of stachyose and/or raffinose within cells of the plant.

30. The method of paragraph 17, wherein the gene product is associated with snetDT1.

31. The method of paragraph 17, wherein the gene product is associated with snetDT2.

32. The method of any of paragraphs 17-31, further comprising propagating the plant to seed and collecting the seeds.

33. The method of paragraph 32, further comprising growing new plants from the collected seeds.

34. A method of cultivating a plant, comprising (A) obtaining a plant that has been transgenically modified to constitutively express a gene product that confers drought tolerance on the plant; and (B) reducing the amount of water used to grow the plant, relative to the amount of water used to grow the same plant in the absence of the transgenic modification.

35. The method of paragraph 34, wherein the step of reducing the amount of water includes growing the plant without artificial irrigation.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

### REFERENCES

1. Shen-Miller, J. et al. Long-living lotus: germination and soil {gamma}-irradiation of centuries-old fruits, and cultivation, growth, and phenotypic abnormalities of offspring. Am J Bot 89, 236-47 (2002).
2. Salton, S. et al. Germination, genetics, and growth of an ancient date seed. Science 320, 1464 (2008).
3. Leino, M. W. & Edqvist, J. Germination of 151-year old Acacia spp. seeds. Genetic Resources and Crop Evolution 57, 741-746 (2010).
4. Oliver, M. J., Tuba, Z. & Mishler, B. The evolution of vegetative desiccation tolerance in land plants. Plant Ecology 151, 85-100 (2000).
5. Oliver, M. J., Velten, J. & Mishler, B. D. Desiccation tolerance in bryophytes: a reflection of the primitive strategy for plant survival in dehydrating habitats? Integr Comp Biol 45, 788-99 (2005).
6. Mishler, B. D. & Churchill, S. P. Transition to a land flora: Phylogenetic relationships of the green algae and bryophytes. Cladistics 1, 305-328 (1985).
7. Verdier, J. et al. A regulatory network-based approach dissects late maturation processes related to the acquisition of desiccation tolerance and longevity of Medicago truncatula seeds. Plant Physiol 163, 757-74 (2013).
8. Righetti, K. et al. Inference of Longevity-Related Genes from a Robust Coexpression Network of Seed Maturation Identifies Regulators Linking Seed Storability to Biotic Defense-Related Pathways. Plant Cell 27, 2692-708 (2015).
9. Manfre, A. J., LaHatte, G. A., Climer, C. R. & Marcotte, W. R., Jr. Seed dehydration and the establishment of desiccation tolerance during seed maturation is altered in the Arabidopsis thaliana mutant atem6-1. Plant Cell Physiol 50, 243-53 (2009).
10. Delahaie, J. et al. LEA polypeptide profiling of recalcitrant and orthodox legume seeds reveals ABI3-regulated LEA protein abundance linked to desiccation tolerance. J Exp Bot 64, 4559-73 (2013).
11. Wehmeyer, N. & Vierling, E. The Expression of Small Heat Shock Proteins in Seeds Responds to Discrete Developmental Signals and Suggests a General Protective Role in Desiccation Tolerance. Plant Physiology 122, 1099-1108 (2000).
12. Bailly, C. Active oxygen species and antioxidants in seed biology. Seed Science Research 14, 93-107 (2004).
13. Baud, S., Boutin, J., Miguel, M., Lepiniec, L. & Rochat, C. An integrated overview of seed development in Arabidopsis thaliana ecotype WS. Plant Physiology and Biochemistry 40, 151-160 (2002).
14. Mene-Saffrane, L., Jones, A. D. & DellaPenna, D. Plastochromanol-8 and tocopherols are essential lipid-soluble antioxidants during seed desiccation and quiescence in Arabidopsis. Proc Natl Acad Sci USA 107, 17815-20 (2010).
15. Chen, M. et al. The effect of transparent TESTA2 on seed fatty acid biosynthesis and tolerance to environmental stresses during young seedling establishment in Arabidopsis. Plant Physiol 160, 1023-36 (2012).
16. Lotan, T. et al. Arabidopsis LEAFY COTYLEDON1 Is Sufficient to Induce Embryo Development in Vegetative Cells. Cell 93, 1195-1205 (1998).
17. Nambara, E., Naito, S. & McCourt, P. A mutant of Arabidopsis which is defective in seed development and storage protein accumulation is a new abi3 allele. The Plant Journal 2, 435-441 (1992).
18. Giraudat, J. et al. Isolation of the Arabidopsis ABI3 gene by positional cloning. The Plant Cell 4, 1251-61 (1992).
19. LuerRen, H., Kirik, V., Herrmann, P. & Misera, S. FUSCA3encodes a protein with a conserved VP1/ABI3-like B3 domain which is of functional importance for the regulation of seed maturation in Arabidopsis thaliana. The Plant Journal 15, 755-764 (1998).
20. Stone, S. L. et al. LEAFY COTYLEDON2 encodes a B3 domain transcription factor that induces embryo development. Proceedings of the National Academy of Sciences 98, 11806-11811 (2001).
21. Keith, K., Kraml, M., Dengler, N. G. & McCourt, P. fusca3: A Heterochronic Mutation Affecting Late Embryo Development in Arabidopsis. The Plant Cell 6, 589-600 (1994).
22. Meinke, D. W., Franzmann, L. H., Nickle, T. C. & Yeung, E. C. Leafy Cotyledon Mutants of Arabidopsis. The Plant Cell 6, 1049-1064 (1994).
23. To, A. et al. A network of local and redundant gene regulation governs Arabidopsis seed maturation. Plant Cell 18, 1642-51 (2006).
24. Roscoe, T. T., Guilleminot, J., Bessoule, J. J., Berger, F. & Devic, M. Complementation of Seed Maturation Phenotypes by Ectopic Expression of ABSCISIC ACID INSENSITIVE3, FUSCA3 and LEAFY COTYLEDON2 in Arabidopsis. Plant Cell Physiol 56, 1215-28 (2015).
25. Fait, A. et al. Arabidopsis Seed Development and Germination Is Associated with Temporally Distinct Metabolic Switches. Plant Physiology 142, 839-854 (2006).
26. McCarthy, D. J., Chen, Y. & Smyth, G. K. Differential expression analysis of multifactor RNA-Seq experiments with respect to biological variation. Nucleic Acids Res 40, 4288-97 (2012).
27. Basso, K. et al. Reverse engineering of regulatory networks in human B cells. Nat Genet 37, 382-90 (2005).
28. Forsthoefel, N. R., Cutler, K., Port, M. D., Yamamoto, T. & Vernon, D. M. PIRLs: a novel class of plant intracellular leucine-rich repeat proteins. Plant Cell Physiol 46, 913-22 (2005).
29. Mu, J. et al. LEAFY COTYLEDON1 Is a Key Regulator of Fatty Acid Biosynthesis in Arabidopsis. Plant Physiology 148, 1042-1054 (2008).
30. Wu, Y. et al. Dual function of Arabidopsis ATAF1 in abiotic and biotic stress responses. Cell Res 19, 1279-90 (2009).
31. Kim, D. H. et al. SOMNUS, a CCCH-type zinc finger protein in Arabidopsis, negatively regulates light-dependent seed germination downstream of PIL5. Plant Cell 20, 1260-77 (2008).

What is claimed:

1. A transgenic plant, comprising a plant expressing in vegetative tissues an exogenous gene, wherein the exogenous gene encodes a transcription factor that confers drought resistance on the plant.

2. The transgenic plant of claim 1, wherein the exogenous gene is involved in seed development or production in wild-type plants.

3. The transgenic plant of claim 1, wherein the transcription factor is selected from the group consisting of PLATZ1, PLATZ2, ANAC089, ERF12, and AGL67.

4. The transgenic plant of claim 3, wherein the transcription factor is PLATZ1.

5. The transgenic plant of claim 1, the exogenous gene being a first exogenous gene, the plant further expressing a second exogenous gene, the second exogenous gene also conferring drought resistance on the plant.

6. The transgenic plant of claim 5, wherein the first exogenous gene and the second exogenous gene encode distinct transcription factors.

7. The transgenic plant of claim 6, wherein each distinct transcription factor is selected from the group consisting of PLATZ1, PLATZ2, ANAC089, ERF12, and AGL67.

8. The transgenic plant of claim 1, further comprising a constitutive promoter that controls expression of the exogenous gene.

9. The transgenic plant of claim 1, further comprising an inducible promoter that controls expression of the exogenous gene, wherein the inducible promoter causes expression of the exogenous gene when the transgenic plant is experiencing water stress.

10. The transgenic plant of claim 1, further comprising a modification of the promoter that controls expression of the endogenous gene orthologous to PLATZ1, PLATZ2, ANAC089, ERF12, and AGL67, wherein the modification of the promoter causes expression of the endogenous gene when the transgenic plant is experiencing water stress.

11. A method of conferring drought resistance on a plant, comprising modifying cells of the plant to constitutively express a gene encoding a gene product that is normally expressed mainly during seed production.

12. The method of claim 11, wherein the gene product is a transcription factor.

13. The method of claim 12, wherein the transcription factor is selected from the group consisting of PLATZ1, PLATZ2, ANAC089, ERF12, and AGL67.

14. The method of claim 11, wherein the gene is an exogenous gene.

15. The method of claim 11, wherein modifying cells of the plant includes introducing a constitutive promoter into the cells upstream from the gene, such that the gene is constitutively expressed.

16. The method of claim 11, wherein modifying cells of the plant includes introducing an inducible promoter into the cells upstream from the gene, such that the gene is inducibly expressed when the transgenic plant is experiencing water stress.

17. The method of claim 11, wherein the gene product leads to the accumulation of stachyose and/or raffinose within cells of the plant.

18. The method of claim 11, further comprising propagating the plant to seed and collecting the seeds.

19. A method of cultivating a plant, comprising:
obtaining a plant that has been transgenically modified to constitutively express a gene product that confers drought tolerance on the plant; and
reducing the amount of water used to grow the plant, relative to the amount of water used to grow the same plant in the absence of the transgenic modification.

20. The method of claim 19, wherein the step of reducing the amount of water includes growing the plant without artificial irrigation.
